(19) United States
(12) Patent Application Publication
(10) Pub. No.: US 2011/0054029 A1
(43) Pub. Date: Mar. 3, 2011

(54) WATER-SOLUBLE DIETARY FATTY ACIDS

(76) Inventor: Eric H. Kuhrts, Bodega, CA (US)

(21) Appl. No.: 12/823,945

(22) Filed: Jun. 25, 2010

Related U.S. Application Data
(60) Provisional application No. 61/220,955, filed on Jun. 26, 2009.

(51) Int. Cl. A61K 31/202; A61P 35/00; A61P 3/04; A61P 9/00; A61P 3/06; A61P 25/00; A61P 27/02

(52) U.S. Cl. 514/560

(57) ABSTRACT

Water-soluble dietary fatty acid formulations, solutions, and methods for increasing the water solubility and/or bioavailability of dietary fatty acids, as well as methods for treating various diseases are disclosed.

### BACKGROUND

Dietary or nutritional fatty acids are a family of unsaturated fatty acids that include the omega-3 fatty acids such as eicosapentaenoic acid (EPA) and docosahexaenoic acid (DHA), as well as omega-6 and omega-9 fatty acids. One of the primary sources for the omega-3 fatty acids is fish oil; however, omega-3 fatty acids can also be obtained from botanical sources and algae. The cardiovascular and other health benefits of these fatty acids are known in addition to their general nutritional benefits. Due to the increased awareness of the health benefits of the omega-3 class of fatty acids, dietary food supplements of fish oil and flax oil have become popular, and a number of food companies have added fish oils to food and beverage products.

Until recently, deodorized fish oils with virtually no fishy taste or smell have not been available. However, with the availability of deodorized fish oils, it is now possible to make beverages containing omega-3 fatty acids, or fish oil, but the solubility of the oil in water containing beverages is a problem. Thus, it would be desirable to provide a formulation of nutritional fatty acids that are soluble in water containing beverages, or a water-soluble omega-3 fatty acid formulation that could be consumed as a beverage. It would also be desirable to have a clear beverage that is not cloudy or opaque. In addition, it would also be desirable to have a process or method of making such formulations.

Furthermore, it is noted that consumption of nutritional or dietary fatty acids have been identified with many health benefits, having the potential to impact numerous diseases such as cardiovascular, neurological, immune function, and arthritis. In order for any therapeutic molecular substance to be efficiently transported through the gastrointestinal tract, enter the blood, and eventually reach the organs and cells inside the body, the molecule should be dissolvable in the aqueous phase of the intestinal fluid. Without an acceptable amount of dissolution, the drug would mostly pass through the GI-tract. Fats or oils (lipids) can become more absorbable if they are emulsified in the stomach as part of digestion. This process involves the generation of a lipid-water interface and an interaction between water-soluble lipases and insoluble lipids or fats. The absorption of lipids is enhanced greatly by this process. By already forming a lipid-water complex through a pre-existing water-soluble formulation, the bioavailability or absorption of lipids such as dietary fatty acids, can be enhanced. The problem is that nutritional fatty acids such as omega-3 fatty acids are virtually insoluble in water, and if added to beverages as a cloudy emulsion, suspension, or oil in water mixture, they are less than satisfactory to consumers for consumption.

Due to the many desirable properties of nutritional or dietary fatty acids, it would be advantageous to provide a more water-soluble formulation and/or enhanced bioavailability formulation of these fatty acids for in vivo use.

### SUMMARY

This disclosure relates to unique pharmaceutical compositions comprising water-soluble formulations of dietary or nutritional fatty acids. Specifically, a water-soluble dietary fatty acid gel formulation can comprise from 1 wt % to 75 wt % of dietary fatty acid; and from 25 wt % to 99 wt % of non-ionic surfactant. Further, a method of delivering a dietary fatty acid to a subject can comprise administering the water-soluble dietary fatty acid gel formulation to a subject such that the dietary fatty acid is more bioavailable then when the same amount of dietary fatty acid is delivered alone.

In another embodiment, a dietary fatty acid solution can comprise from 0.1 wt % to 94.9 wt % of water; from 0.1 wt % to 35 wt % of dietary fatty acid; and from 5 wt % to 75 wt % of non-ionic surfactant. In one embodiment, the non-ionic surfactant can be present at a concentration to render the dietary fatty acid water-soluble forming a clear solution. Further, a method of delivering a dietary fatty acid to a subject can comprise administering the dietary fatty acid solution to a subject such that the dietary fatty acid is more bioavailable then when the same amount of dietary fatty acid is delivered alone.

A method of dissolving dietary fatty acids in water can comprise the steps of combining a dietary fatty acid with a warm, well mixed non-ionic surfactant to form a surfactant-dietary fatty acid mixture; and continuously mixing the surfactant-dietary fatty acid mixture with water at least as slowly as necessary to solubilize the dietary fatty acid.

Additionally, a method of enhancing the bioavailability of a dietary fatty acid in a subject can comprise dissolving a surfactant-dietary fatty acid mixture in water as described above.

### DETAILED DESCRIPTION

The abbreviations used herein have their conventional meaning within the chemical and biological arts.

“Dietary fatty acids” as used herein, includes nutritional fatty acids, omega-3 fatty acids derived from natural sources such as fish, botanical sources such as chia sage or Salvia hispanica, or flax sources derived from linseed, or which are produced synthetically. The following is a list of omega-3 fatty acids (Table 1) followed by a list of botanical extracts of omega-3 fatty acids (Table 2). These lists are exemplary only, and are not considered to be limiting.

TABLE 1
List of several common n-3 fatty acids found in nature

| Common Name | Lipid Name | Chemical Name |
|---|---|---|
| — | 16:3 (n-3) | all-cis-7,10,13-hexadecatrienoic acid |
| Alpha-Linolenic acid (ALA) | 18:3 (n-3) | all-cis-9,12,15-octadecatrienoic acid |
| Stearidonic acid (STD) | 18:4 (n-3) | all-cis-6,9,12,15-octadecatetraenoic acid |
| Eisosatrienoic acid (ETE) | 20:3 (n-3) | all-cis-11,14,17-eicosatrienoic acid |
| Eicosatetraenoic acid (ETA) | 20:4 (n-3) | all-cis-8,11,14,17-eicosatrienoic acid |
| Eicosapentaenoic acid (EPA) | 20:5 (n-3) | all-cis-5,8,11,14,17-eicosapentaenoic acid |
| Docosapentaenoic acid (DPA), | 22:5 (n-3) | all-cis-7,10,13,16,19-docosapentaenoic acid |
| Clupanodonic acid | | |
| Docosahexaenoic acid (DHA) | 22:6 (n-3) | all-cis-4,7,10,13,16,19-docosahexaenoic acid |
| Tetracosapentaenoic acid | 24:5 (n-3) | all-cis-9,12,15,18,21-docosahexaenoic acid |
| Tetracosahexaenoic acid (Nisinic | 24:6 (n-3) | all-cis-6,9,12,15,18,21-tetracosenoic acid |
| Acid) | | |

TABLE 2
Sources of botanical extracts of omega-3 fatty acids

| Common Name | Alternative Name | Linnaean Name | % n-3 |
|---|---|---|---|
| Chia | Chia sage | Salvia hispanica | 64 |
| Kiwifruit | Chinese gooseberry | Actinidia chinensis | 62 |
| Perilla | Shiso | Perilla frutescens | 58 |
| Flax | Linseed | Linum usitatissimum | 55 |
| Lingonberry | Cowberry | Vaccinium vitis-idaea | 49 |
| Camelina | Gold-of-pleasure | Camelina sativa | 36 |
| Purslane | Portulaca | Portulaca oleracea | 35 |
| Black Raspberry | — | Rubus occidentalis | 33 |

Dietary Fatty Acids containing omega-3 fatty acids may also be derived from algae such as Crypthecodinium cohnii and Schizochytrium, which are rich sources of DHA, or brown algae (kelp) for EPA. They may also include conjugated linoleic acid (CLA), omega-6 fatty acids, and omega-9 fatty acids, such as linolenic acid, linoleic acid (18:2), and gamma linolenic acid (GLA, 18:3).

A “non-ionic surfactant,” as used herein, is a surface-active agent that tends to be non-ionized (i.e. uncharged) in neutral solutions (e.g. neutral aqueous solutions).

The term “treating” refers to any indicia of success in the treatment or amelioration of an injury, pathology or condition, including any objective or subjective parameter such as abatement, remission, diminishing of symptoms; making the injury, pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters, including the results of a physical examination, neuropsychiatric exams, and/or a psychiatric evaluation. Also, treating includes preventative treatment such as promoting the general health of body systems, such as heart or other organ health, etc.

As used herein, the term “cancer” refers to all types of cancer, neoplasm, or malignant tumors found in mammals, including leukemia, carcinomas and sarcomas. Exemplary cancers include cancer of the brain, breast, cervix, colon, head and neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, euroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

“Patient” or “subject” refers to a mammalian subject, including human.

As used herein, the term “titration” or “titrate” means the slow addition of a compound or solution to a liquid while mixing. The rate at which the compound or solution is added should not exceed a certain threshold, or the clear nature and viscosity of the solute is lost. Slow addition can be as a drizzle or drop by drop, but in no case should equal large volumes. Slow addition can be specified as a percent of the volume it is being added to per second or per minute, for example 5 mL per second to 100 mL water, or 5 wt % addition per second or minute of the content being added to water or water containing beverage.

As used herein, the term “clear aqueous solution” in reference to a solution containing dietary fatty acid means a water containing solution (e.g. a beverage) that is free of visible particles of undissolved dietary fatty acid. In accordance with some embodiments, the clear aqueous solution is not a dispersion, and not a suspension, and remains clear upon sitting undisturbed for 1 hour or more. Often, very small micelles are formed that are not visible, and thus, the solution is clear.

The term “water-soluble” herein refers to the solubilization or very fine dispersion of dietary fatty acids so that they are not visible to the naked eye in solution. Often, in the formulations of the present disclosure, the fatty acids can form micelles in water with a non-ionic surfactant barrier, and the micelles can be smaller than about 100 nm in size, and often are about 15 nm to about 30 nm in size. Thus, whether the dietary fatty acids are strictly dissolved or merely so finely dispersed that the solution they form within is clear, this is still considered to be “water-soluble” in accordance with embodiments of the present disclosure.

### Water-Soluble Formulations

It has been discovered that non-ionic surfactants can be used to increase the solubility and/or bioavailability of dietary fatty acids when combined appropriately. Thus, non-ionic surfactants can be used to form fatty acid gel formulations that are highly water-soluble.

In one aspect, the present disclosure provides a water-soluble formulation including a dietary fatty acid, and a non-ionic surfactant. In some embodiments, the water-soluble formulation does not include a vegetable oil suspension or visible macro-micelles (micelles visible to the naked eye) in water. In other embodiments, the water-soluble formulation does not include an alcohol (e.g. the dietary fatty acid is not first dissolved in alcohol and then added to water) or other additives that would otherwise enhance the solubility of the dietary fatty acids.

In accordance with this, a water-soluble dietary fatty acid gel formulation can comprise or consist essentially of from 1 wt % to 75 wt % of dietary fatty acid; and from 25 wt % to 99 wt % of non-ionic surfactant. In one embodiment, the gel formulation can be soluble in water and forms a clear solution at a weight ratio of 1:3 (gel to water). In another embodiment, the gel formulation can be soluble in water and forms a clear solution at a weight ratio of 1:1. In still another embodiment, the dietary fatty acid can be present at from 5 wt % to 60 wt %, and the non-ionic surfactant can be present at from 40 wt % to 95 wt %.

A dietary fatty acid solution can also comprise or consist essentially of from 0.1 wt % to 94.9 wt % of water; from 0.1 wt % to 35 wt % of dietary fatty acid; and from 5 wt % to 75 wt % of non-ionic surfactant. In one embodiment, the water can be present at from 15 wt % to 75 wt %; the dietary fatty acid can be present at from 2 wt % to 20 wt %, and the non-ionic surfactant can be present at from 20 wt % to 50 wt %. In one embodiment, the non-ionic surfactant can be present at a concentration to render the dietary fatty acid water-soluble forming a clear solution.

In accordance with these embodiments the dietary fatty acids can be nutritional fatty acids, omega-3 fatty acids derived from natural sources such as fish, botanical sources such as chia sage or Salvia hispanica, or flax sources derived from linseed, or which are produced synthetically. Exemplary omega-3 fatty acids are set forth in Table 1, and a list of botanical extracts of omega-3 fatty acids are set forth in Table 2. Furthermore, it is noted that dietary fatty acids containing omega-3 fatty acids may also be derived from algae such as Crypthecodinium cohnii and Schizochytrium, which are rich sources of DHA, or brown algae (kelp) for EPA. They may also include conjugated linoleic acid (CLA), omega-6 fatty acids, and omega-9 fatty acids, such as linolenic acid, linoleic acid (18:2), and gamma linolenic acid (GLA, 18:3). Other dietary fatty acids not listed herein can also be used, depending on the desired result to be achieved.

Useful non-ionic surfactants that can be used include, for example, non-ionic water-soluble mono-, di-, and tri-glycerides; non-ionic water-soluble mono- and di-fatty acid esters of polyethyelene glycol; non-ionic water-soluble sorbitan fatty acid esters (e.g. sorbitan monooleates such as SPAN 80 and TWEEN 20 (polyoxyethylene 20 sorbitan monooleate)); polyglycolyzed glycerides; non-ionic water-soluble triblock copolymers (e.g. poly(ethyleneoxide)/poly-(propyleneoxide)/poly(ethyleneoxide) triblock copolymers such as poloxamer 406 (PLURONIC F-127), and derivatives thereof.

Examples of non-ionic water-soluble mono-, di-, and tri-glycerides include propylene glycol dicarpylate/dicaprate (e.g. Miglyol 840), medium chain mono- and diglycerides (e.g. Capmul and ImwitoR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), polyethylene glycol 660 hydroxystearate, and derivatives thereof.

Non-ionic water-soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include Labrafil M-1944CS, Labrafil M-2125CS, Labrasol, SOFTIGEN, and GELUCIRE.

In some embodiments, the non-ionic surfactant is a glycerol-polyethylene glycol oxystearate, or derivative thereof. These compounds may be synthesized by reacting either castor oil or hydrogenated castor oil with varying amounts of ethylene oxide. Macrogolglycerol ricinoleate is a mixture of 83 wt % relatively hydrophobic and 17 wt % relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate (glycerol-polyethylene glycol oxysterate) is a mixture of approximately 75 wt % relatively hydrophobic of which a major portion is glycerol polyethylene glycol 12-oxystearate.

In some embodiments, the water-soluble formulations include the dietary fatty acid, and glycerol-polyethylene glycol oxystearate, to form a transparent water-soluble formulation, which means that the formulation can be clearly seen through with the naked eye, but may be optionally colored. The transparent water-soluble formulation can be solvated in water to form a clear solution. In some embodiments, the transparent water-soluble formulations do not contain particles (e.g. particles of undissolved dietary fatty acid) visible to the naked eye. In certain embodiments, light may be transmitted through the transparent water-soluble formulations without diffusion or scattering. Thus, in some embodiments, the transparent water-soluble formulations are not opaque, cloudy or milky-white.

In some embodiments, the water-soluble formulation is a non-alcoholic formulation, which indicates that the formulation that does not include (or includes only in trace amounts) methanol, ethanol, propanol or butanol. In other embodiments, the formulation does not include (or includes only in trace amounts) ethanol.

In some embodiments, the formulation can be a non-aprotic solvated formulation, meaning that water-soluble aprotic solvents are absent or are included only in trace amounts. Water-soluble aprotic solvents are water-soluble non-surfactant solvents in which the hydrogen atoms are not bonded to an oxygen or nitrogen and therefore cannot donate a hydrogen bond.

In some embodiments, the water-soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water-soluble formulation does not include (or includes only in trace amounts) dimethyl sulfoxide. Thus, in some embodiments, the water-soluble formulation does not include DMSO. In a related embodiment, the water-soluble formulation does not include DMSO or ethanol.

In still other embodiments, the water-soluble formulation does not include (or includes only in trace amounts) a non-polar aprotic solvent. Non-polar aprotic solvents are aprotic solvents whose molecules exhibit a molecular dipole of approximately zero. Examples include hydrocarbons, such as alkanes, alkenes, and alkynes.

The water-soluble formulation of the present invention includes formulations dissolved in water (i.e. aqueous formulations). In some embodiments, the water-soluble formulation forms a transparent water-soluble formulation when added to water. Thus, in accordance with some embodiments of the present disclosure, because of the nature of the water-soluble dietary fatty acid gel formulations prepared herein, often, only water and optionally a small amount of a stabilizing agent is all that is used to form the dietary fatty acid solutions of the present disclosure, e.g., alcohol, aprotic solvents (polar or non-polar), etc., are not required for solvating the dietary fatty acids.

In some embodiments, the water-soluble formulation consists essentially of dietary fatty acid and a non-ionic surfactant. Where a water-soluble formulation “consists essentially of” dietary fatty acid and a non-ionic surfactant, the formulation includes the dietary fatty acid, the non-ionic surfactant, and optionally additional components widely known in the art to be useful in neutraceutical formulations, such as preservatives, taste enhancers, colors, buffers, water, etc., which do not impact the basic solubility of the formulation, i.e. no additional organic solvating solvents are required.

In some embodiments, the water-soluble formulation is a water-solubilized formulation, meaning that the dietary fatty acid and a non-ionic surfactant are admixed with water (e.g. a water containing liquid) to form the solutions of the present disclosure, but does not include organic solvents (e.g. ethanol or other alcohol or solvating solvent). In some embodiments, the water solubilized formulation a transparent water-soluble formulation.

### Method

In another aspect of the present invention is described a method of producing the water-soluble fatty acid formulations. Simply warming and mixing the dietary fatty acids with a non-ionic surfactant (such as glycerol-polyethylene glycol oxystearate or other similar non-ionic surfactant) will not result in a clear water-soluble solution unless it is added appropriately. Instead, a semi-solid gel-like cloudy or milky, high viscosity solution is obtained by simple mixing. This waxy, cloudy, high viscosity gel is not suitable for forming clear solutions in water or beverages. It becomes a solidified milky white mass. By slowly titrating or adding the dietary fatty acid into the warm non-ionic surfactant while mixing, a clear solution can be obtained.

More specifically, a method of dissolving dietary fatty acids in water can comprise the steps of combining a dietary fatty acid with a warm, well mixed non-ionic surfactant to form a surfactant-dietary fatty acid mixture; and continuously mixing the surfactant-dietary fatty acid mixture with water at least as slowly as necessary to solubilize the dietary fatty acid. In certain specific embodiments, the warm, well mixed non-ionic surfactant is prepared by the preliminary step of heating the surfactant to a temperature of about 90° F. to about 200° F. while mixing until clear. In another specific embodiment, the combining step includes adding the dietary fatty acid to the non-ionic surfactant slowly and stirring until thoroughly mixed. The dietary fatty acid can be sufficiently dispersed or dissolved in the surfactant so that a resultant solution contains no visible micelles or particles of dietary fatty acid. For example, the mixing step can include slowly adding the surfactant-dietary fatty acid mixture to warm water at a rate not to exceed 5 vol % of the water per second. Furthermore, the step of heating the water-soluble non-ionic surfactant can include the step of stirring or mixing during the heating step.

The rate at which the dietary fatty acid is added to the warm surfactant, and the temperature of the surfactant can be aided by carrying out the process appropriately for a desired result, e.g., forming a clear solution. For example, in some embodiments, the surfactant should not be below a certain temperature or above a certain temperature. Likewise, if the dietary fatty acid gel mixture is added to the water too fast, a solid gel-like mass will result. The non-ionic surfactant should typically also be stirred thoroughly to remove bubbles (oxygen), and until clear. Once the dietary fatty acid has been added to the surfactant, it is stirred for at least 10 minutes, or more, and typically for about 1 hour.

In further detail, when adding the water-soluble dietary fatty acid gel formulation to water, the formulation should be added at a rate not to exceed 5 mL per second to a volume of water of 100 mL, or not more than 5 vol % of the water per second of the volume of water it is being added to. The rate of addition depends on the volume of water. Further, the water can be stirred continuously while the addition of the dietary fatty acid gel is being slowly added. The solution may be heated to increase solubility, if desired or necessary. That being said, the heating temperature is typically selected to avoid chemical breakdown of the dietary fatty acid and/or non-ionic surfactant. The temperature of the dietary fatty acid gel (dietary fatty acid/non-ionic surfactant) should not typically exceed 200° F., and the water temperature should also not typically exceed 200° F. Ideally, the temperature of both should be maintained at from 100 to 150° F., and in one embodiment, the water can optionally be maintained at about 100° F. while slowly adding the dietary fatty acid gel mixture. In some embodiments, the resulting solution is a water-soluble formulation or transparent water-soluble formulation as described above. For example, the resulting solution may be a water-soluble formulation that is a crystal clear solution, with no particles visible to the naked eye.

The present disclosure also provides a method of delivering a dietary fatty acid to a subject, comprising administering the formulation or solution described herein to a subject such that the dietary fatty acid is more bioavailable than when the same amount of dietary fatty acid is delivered alone. Administration routes will be described in detail hereinafter, but suffice it to say that any administration route can be used that is effective for treating a disease or providing a health benefit, e.g., oral, mucosal, ocular, parenteral, or topical delivery.

Thus, the present disclosure can provide a method of treating cancer, obesity, diabetes, cardiovascular disease, dyslipidaemia, age-related macular degeneration (e.g. vision loss associated with age-related macular degeneration), high cholesterol, retinopathy (e.g. diabetic retinopathy), or a neurological disease in subject in need of such treatment. The method includes administering to the subject an effective amount of the water-soluble formulations disclosed herein. It is noted that thought these diseases are provided in a common list, they are not equivalent diseases and should be considered herein as if each are listed separately.

In another aspect, the present invention provides a method for enhancing the bioavailability of dietary fatty acid. The method includes combining dietary fatty, and a non-ionic surfactant to form a surfactant-dietary fatty acid mixture. The surfactant-dietary fatty acid mixture may be administered to the subject thereby enhancing the bioavailability of the dietary fatty acid. The bioavailability is enhanced compared to the bioavailability of dietary fatty acid in the absence of non-ionic surfactant.

### Dosages and Dosage Forms

The amount of dietary fatty acid adequate to treat a disease or provide a health benefit can be defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient and disease or condition treated.

Single or multiple administrations of dietary fatty acid formulations can be administered depending on the dosage and frequency as required and tolerated by the patient. The formulations should provide a sufficient quantity of active agent to effectively treat the disease state, or to provide the appropriate health benefit. Lower dosages can be used, particularly when the dietary fatty acid is administered to an anatomically secluded site in contrast to administration orally, into the blood stream, into a body cavity or into a lumen of an organ. Higher dosages can be used in topical administration. Actual methods for preparing parenterally administrable dietary fatty acid formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

In some embodiments, the dietary fatty acid is present in the water-soluble dietary gel formulation at a concentration of 1 wt % to 75 wt %, or alternatively, at from 5 wt % to 50 wt %, 10 wt % to 35 wt %, or 20 wt % to 25 wt %. The dietary fatty acid may also be present as a solution in a ready to drink beverage formulation at a concentration from 0.1 mg/mL to 10 mg/mL, or alternatively, from 0.5 mg/mL to 5 mg/mL. If making a concentrate to be added to additional water, the concentration can be from 10 to 125 mg/mL, for example. These ranges are not intended to be limiting, but rather provide guidelines for preparing ready to drink formulations, as well as concentrates. It is noted that there can be a maximum concentration for achieving a crystal clear solution, if a clear solution is desired.

The water-soluble formulation can also be in the form of a pharmaceutical composition. The pharmaceutical composition may include dietary fatty acid, a non-ionic surfactant, and a pharmaceutically acceptable excipient. After a pharmaceutical composition including dietary fatty acid of the present disclosure has been formulated in an acceptable carrier, it can be placed in an appropriate container and labeled for treatment of an indicated condition. For administration of dietary fatty acid, such labeling would include, for example, instructions concerning the amount, frequency and method of administration.

Any appropriate dosage form is useful for administration of the water-soluble formulation of the present disclosure, such as oral, parenteral, mucosal, ocular, and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. Examples of liquid formulations include drops, sprays, aerosols, emulsions, lotions, suspensions, drinking solutions, gargles, and inhalants. The formulations of the present disclosure can also be administered by injection, that is, intravenously, intramuscularly, intracutaneously, subcutaneously, intraduodenally, or intraperitoneally. Also, the formulations described herein can be administered by inhalation, for example, intranasally. Additionally, the formulations of the present invention can be administered topically, such as transdermally. The formulations can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions from the formulations of the present disclosure, pharmaceutically acceptable carriers can be either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain dietary fatty acid mixed with a filler or binders such as lactose or starches, lubricants such as talc or magnesium stearate, and, optionally, stabilizers. In soft capsules, dietary fatty acid may be dissolved or suspended in suitable liquids, such as fatty oils, liquid paraffin, or liquid polyethylene glycol with or without stabilizers, or alternatively, may be encapsulated as the water-soluble dietary fatty acid gel formulation (prior to addition of water).

For preparing suppositories, a low melting wax, such as a mixture of fatty acid glycerides or cocoa butter, can be first melted and the active component dispersed homogeneously therein, such as by stirring. The molten homogeneous mixture is then poured into convenient sized molds, allowed to cool, and thereby to solidify.

Liquid form preparations include solutions, suspensions, beverages, and emulsions, for example, water or water/propylene glycol solutions. For parenteral injection, liquid preparations can be formulated in solution in aqueous polyethylene glycol solution or other suitable solution for injection.

Aqueous solutions and beverages suitable for oral use can be prepared by dissolving the water-soluble dietary fatty acid gel formulation in water and adding suitable colorants, flavors, stabilizers, and thickening agents as desired. Aqueous solutions or suspensions suitable for oral use can be made by dispersing the active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which may be converted, shortly before use, to liquid form preparations for oral administration. Such liquid forms include solutions, suspensions, and emulsions. These preparations may contain, in addition to the dietary fatty acid, colorants, flavors, stabilizers, buffers, artificial and natural sweeteners, dispersants, thickeners, solubilizing agents, and the like.

Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the invention can be delivered transdermally, by a topical route, formulated as applicator sticks, solutions, suspensions, emulsions, gels, creams, ointments, pastes, jellies, paints, powders, and aerosols.

The formulations can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1 wt % to 2 wt % sucrose, 2 wt % to 7 wt % mannitol at a pH range of 4.5 to 5.5, that is combined with buffer prior to use.

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the dietary fatty acid, dietary fatty acid metabolite or slat thereof into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

### Assays

Subject non-ionic surfactants may be assayed for their ability to solubilize dietary fatty acid using any appropriate method. Typically, a non-ionic surfactant is warmed and contacted with the dietary fatty acid and mixed mechanically and/or automatically using a shaker, vortex, or sonicator device. Water may be optionally added, for example, where the dietary fatty acid and/or surfactant are in powder form. The solution is heated to increase solubility. The heating temperature is selected to avoid chemical breakdown of the dietary fatty acid or non-ionic surfactant. The surfactant or dietary fatty acid should typically not be heated above 200° F., and preferably not more than 150° F.

The resulting solution may be visually inspected for colloidal particles to determine the degree of solubility of the dietary fatty acid. Alternatively, the solution may be filtered and analyzed to determine the degree of solubility. For example, a spectrophotometer may be used to determine the concentration of dietary fatty acid present in the filtered solution. Typically, the test solution is compared to a positive control containing a series of known quantities of pre-filtered dietary fatty acid solutions to obtain a standard concentration versus UV/vis absorbance curve. Alternatively, high performance liquid chromatography may be used to determine the amount of dietary fatty acid in solution.

High throughput solubility assay methods are well known in the art. Typically, these methods involve automated dispensing and mixing of solutions with varying amounts of non-ionic surfactants, dietary fatty acid, and optionally other co-solvents. The resulting solutions may then be analyzed to determine the degree of solubility using any appropriate method as discussed above.

The Millipore MultiScreen Solubility filter Plate® with modified track-etched polycarbonate, 0.4 μm membrane is a single-use, 96-well product assembly that includes a filter plate and a cover. The device is intended for processing aqueous solubility samples in the 100-300 μL volume range. The vacuum filtration design is compatible with standard, microtiter plate vacuum manifolds. The plate is also designed to fit with a standard, 96-well microtiter receiver plate for use in filtrate collection. The MultiScreen Solubility filter Plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of dietary fatty acid solubility by mixing, incubating and filtering a solution in the MultiScreen Solubility filter plate. After the filtrate is transferred into a 96-well collection plate using vacuum filtration, it is analyzed by UV/vis spectroscopy to determine solubility. Additionally, LC/MS or HPLC can be used to determine compound solubility, especially for compounds with low UV/Vis absorbance and/or compounds with lower purity. For quantification of aqueous solubility, a standard calibration curve may be determined and analyzed for each compound prior to determining aqueous solubility.

Test solutions may be prepared by adding an aliquot of concentrated a given compound. The solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well are transferred from the collection plate to a 96-well UV analysis plate and diluted with 40 μL/well of acetonitrile. The UV/vis analysis plate is scanned from 260-500 nm with a UV/vis microplate spectrometer to determine the absorbance profile of the test compound.

Thus, one skilled in the art may assay a wide variety of non-ionic surfactants to determine their ability of solubilize dietary fatty acid compounds in accordance with embodiments of the present disclosure.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the formulations are equally applicable to the methods of treating disease states described herein. All publications, patents, and patent applications cited herein are hereby incorporated by reference in their entirety for all purposes.

### EXAMPLES

The examples below are meant to illustrate certain embodiments of the disclosure, and are intended not to limit the scope of the invention. It is noted that Lucifer Yellow is from Molecular Probes (Eugene, Oreg.). Hanks buffer and all other chemicals are obtained from Sigma-Aldrich (St. Louis, Mo.).

### Example 1

### Preparation of Omega-3 Gel Formulations (Fish Oil) and Subsequent Aqueous Solutions of Omega-3 Fatty Acids

Water-soluble compositions of omega-3 fatty acids are formulated using the non-ionic surfactant macrogolglycerol hydroxystearate (Glycerol-Polyethylene glycol oxystearate). First, the non-ionic surfactant is heated to about 115° F. and stirred until clear and virtually no bubbles are apparent. A deodorized omega-3 fatty acid fish oil, containing 30 wt % omega-3 fatty acids at room temperature is very slowly added or titrated into the warm macrogolglycerol hydroxystearate until a clear slightly viscous solution is formed containing dissolved omega-3 fatty acids (or “omega-3 gel formulation” or “fatty acid gel formulation”). The omega-3 gel formulation thus comprises 50 g of the macrogolglycerol hydroxystearate and 10 g of omega-3 fatty acids, representing about 17 wt % of the omega-3 fatty acids gel formulation. The omega-3 fatty gel formulation is slowly titrated at a rate of about 1 mL per second to 100 mL of warm water maintained as a mixing vortex with a stirrer at 100 RPM, and maintained at a temperature of about 110° F. until a crystal clear solution is formed. The water is continuously stirred during the addition phase and shortly thereafter after.

As can be seen from the above example, an aqueous solution of solubilized omega-3 fatty acids is achieved by adding the omega-3 fatty acid gel formulation to the warm water, thereby making a water-soluble beverage. More specifically, the aqueous omega-3 fatty acid gel formulation is prepared by maintaining the gel formulation at a temperature of about 115° F. and titrating or adding drop by drop the gel mixture to warm water to form a clear aqueous solution (or very fine dispersion that is visually clear) of omega-3 fatty acids. This aqueous omega-3 fatty acid formulation will not have an undesirable flavor. The aqueous omega-3 fatty acid formulation included water (100 mL), macrogolglycerol hydroxystearate 40 (50 mL), and a deodorized, 30 wt % omega-3 fatty acid fish oil (10 mL), a concentration of omega-3 fatty acids in the aqueous dietary fatty acid formulation is about 6.6 wt % (water containing beverage). A visual inspection confirmed that the solution will be crystal-clear with no visible particles. The aqueous omega-3 fatty acid formulation is analyzed by HPLC to verify its contents.

### Example 2

The solubility of the omega-3 fatty acids in pH 7.4 Hank's Balanced Salt Solution (10 mM HEPES and 15 mM glucose) is compared to the omega-3 gel formulation. At least 1 mg omega-3 fatty acid oil (30 wt % omega-3) as well as 100 mg of omega-3 gel formulation is combined with 1 mL of buffer to make a mg/mL omega-3 oil mixture and a mg/mL omega-3 gel formulation mixture, respectively. The respective mixtures are shaken for 2 hours using a benchtop vortexer and left to stand overnight at room temperature. After vortexing and standing overnight, the omega-3 oil mixture is then filtered through a 0.45-μm nylon syringe filter (Whatman, Cat# 6789-0404) that is first saturated with the sample.

After vortexing and standing overnight, the omega-3 gel formulation mixture is centrifuged at 14,000 rpm for 10 minutes. The filtrate or supernatant is sampled twice, consecutively, and diluted 10, 100, and 10,000-fold in a mixture of 50:50 assay buffer:acetonitrile prior to analysis.

Both mixtures are assayed by LC/MS/MS using electrospray ionization against the standards prepared in a mixture of 50:50 assay buffer:acetonitrile. Standard concentrations ranged from 1.0 μM down to 3.0 nM. Results would indicate a significant difference in solubility between the two formulations.

### Example 3

To test the permeability of dietary fatty acids across Caco-2 cell monolayers, Caco-2 cell monolayers are grown to confluence on collagen-coated, microporous, polycarbonate membranes in 12-well Costar Transwell® plates.

The test article is the aqueous dietary fatty acids formulation, and the dosing concentration is 2 μM in the assay buffer (HBSSg) as in the previous example. Cell monolayers are dosed on the apical side (A-to-B) or basolateral side (B-to-A) and incubated at 37° C. with 5% CO₂ in a humidified incubator. Samples are taken from the donor chamber at 120 minutes, and samples from the receiver chamber are collected at 60 and 120 minutes. Each determination is performed in duplicate. Lucifer yellow permeability is also measured for each monolayer after being subjected to the test article to ensure no damage is inflicted to the cell monolayers during the permeability experiment. Permeability of samples of atenolol, propranolol and digoxin are also measured to compare with the permeability of the dietary fatty acids sample. All samples are assayed for dietary fatty acids, or corresponding comparative compounds, by LC/MS/MS using electrospray ionization. The apparent permeability (Papp) and percent recovery are calculated as is known in the art. Dietary fatty acids permeability results can be presented as by reporting the permeability (10⁻⁶ cm/s) and recovery of Dietary fatty acids across Caco-2 cell monolayers. All monolayers pass the post-experiment integrity control with Lucifer yellow Papp <0.8×10⁻⁶ cm/s.

### Example 4

### Preparation of Omega-3 Gel Formulations (Flax Seed Oil) and Subsequent Aqueous Solutions of Omega-3 Fatty Acids

Five (5) grams of flax seed oil is dissolved in 50 mL of warm Polyethylene Glycol 660 Hydroxystearate by mixing until a clear gel is formed (“omega-3 gel formulation”). The omega-3 gel formulation is then very slowly added to 100 mL of warm distilled water while continuous mixing (e.g., with a paddle suspended and rotating at 100 RPM by slowly adding as a drizzle, or drop-by-drop using a titration apparatus). The omega-3 gel formulation with flax seed oil is added very slowly to the mixing water to avoid solidification of the liquid into a solid gel, or cloudy white mass (e.g., at a rate of 1 mL every 10 seconds or more while stirring continues). A clear solution is formed with no visible particles or micelles.

### Example 5

### Preparation of Omega-3 Gel Formulations (Fish Oil) and Subsequent Aqueous Solutions of Omega-3 Fatty Acids

30 grams of fish oil is dissolved in 50 mL of warm macrogolglycerol hydroxystearate (Glycerol-Polyethylene glycol oxystearate) by mixing until a gel is formed (“omega-3 gel formulation”). The omega-3 gel formulation is then very slowly added to 200 mL of warm distilled water while continuous mixing (e.g., with a paddle suspended and rotating at 100 RPM by slowly adding as a drizzle, or drop-by-drop using a titration apparatus). The omega-3 gel formulation with fish oil is added very slowly to the mixing water to avoid solidification of the liquid into a solid gel, or cloudy white mass (e.g., at a rate of 1 mL every 10 seconds or more while stirring continues). A clear solution is formed with no visible particles or micelles.

What is claimed is:

1. A water-soluble dietary fatty acid gel formulation, comprising:
from 1 wt % to 75 wt % of dietary fatty acid; and
from 25 wt % to 99 wt % of non-ionic surfactant.

2. The formulation of claim 1, wherein the gel formulation is soluble in water and forms a clear solution at a weight ratio of 1:3.

3. The formulation of claim 1, wherein the gel formulation is soluble in water and forms a clear solution at a weight ratio of 1:1.

4. The formulation of claim 1, wherein the dietary fatty acid is an omega-3 fatty acid.

5. The formulation of claim 4, wherein the omega-3 fatty acid is eicosapentaenoic acid (EPA), docosahexaenoic acid (DHA), or a mixture thereof.

6. The formulation of claim 1, wherein the dietary fatty acid is present at a concentration of at least 20 wt %.

7. The formulation of claim 1, wherein the non-ionic surfactant is a non-ionic water-soluble mono-, di-, or tri-glyceride; non-ionic water-soluble mono- or di-fatty acid ester of polyethyelene glycol; non-ionic water-soluble sorbitan fatty acid ester; polyglycolyzed glyceride; non-ionic water-soluble triblock copolymers; derivative thereof; or combinations thereof.

8. The formulation of claim 1, wherein the non-ionic surfactant is a non-ionic water-soluble mono-, di-, or tri-glyceride.

9. The formulation of claim 1, wherein the non-ionic surfactant is glycerol-polyethylene glycol oxystearate.

10. The formulation of claim 1, wherein the non-ionic surfactant is macrogolglycerol ricinoleate, macrogolglycerol hydroxystearate, polyethylene glycol 660 hydroxystearate, or a mixture thereof.

11. The formulation of claim 1, wherein the non-ionic surfactant is polyethylene glycol 660 hydroxystearate.

12. The formulation of claim 1, wherein the formulation is an oral formulation.

13. The formulation of claim 1, wherein the formulation is a mucosal, parenteral, ocular, or topical formulation.

14. The formulation of claim 1, wherein the dietary fatty acid is present at from 5 wt % to 60 wt %, and the non-ionic surfactant is present at from 40 wt % to 95 wt %.

15. The formulation of claim 1, wherein the dietary fatty acid is derived from a fish, algae, or vegetable source.

16. The formulation of claim 1, further comprising a pharmaceutically acceptable excipient or stabilizer.

17. The formulation of claim 1, consisting essentially of the dietary fatty acid and the non-ionic surfactant.

18. A method of delivering a dietary fatty acid to a subject, comprising administering the formulation of claim 1 to a subject such that the dietary fatty acid is more bioavailable than when the same amount of dietary fatty acid is delivered alone.

19. The method of claim 18, wherein the step of administering is by oral, mucosal, ocular, parenteral, or topical delivery.

20. The method of claim 18, wherein the administering is a result of the subject being treated for cancer, obesity, diabetes, cardiovascular disease, dyslipidaemia, age-related macular degeneration, high cholesterol, retinopathy, or a neurological disease.

21. A dietary fatty acid solution, comprising:
from 0.1 wt % to 94.9 wt % of water;
from 0.1 wt % to 35 wt % of dietary fatty acid; and
from 5 wt % to 75 wt % of non-ionic surfactant.

22. The solution of claim 21, wherein the water is present at from 15 wt % to 75 wt %; the dietary fatty acid is present at from 2 wt % to 20 wt %, and the non-ionic surfactant is present at from 20 wt % to 50 wt %.

23. The solution of claim 21, wherein the non-ionic surfactant is present at a concentration to render the dietary fatty acid water-soluble, forming a clear solution.

24. The solution of claim 21, wherein the dietary fatty acid is an omega-3 fatty acid.

25. The solution of claim 24, wherein the omega-3 fatty acid is eicosapentaenoic acid (EPA), docosahexaenoic acid (DHA), or a mixture thereof.

26. The solution of claim 21, wherein the formulation is a non-alcoholic formulation.

27. The solution of claim 21, wherein the formulation is a non-aprotic solvated formulation.

28. The solution of claim 21, wherein the dietary fatty acid is present at a concentration of at least 0.1 mg/mL.

29. The solution of claim 21, wherein the dietary fatty acid is present at a concentration of at least 1 mg/mL.

30. The solution of claim 21, wherein the dietary fatty acid is present at a concentration from 0.1 mg/mL to 10 mg/mL.

31. The solution of claim 21, wherein the dietary fatty acid is present at a concentration from 10 to 125 mg/mL.

32. The solution of claim 21, wherein the non-ionic surfactant is a non-ionic water-soluble mono-, di-, or tri-glyceride; non-ionic water-soluble mono- or di-fatty acid ester of polyethyelene glycol; non-ionic water-soluble sorbitan fatty acid ester; polyglycolyzed glyceride; non-ionic water-soluble triblock copolymers; derivative thereof; or combinations thereof.

33. The solution of claim 21, wherein the non-ionic surfactant is a non-ionic water-soluble mono-, di-, or tri-glyceride.

34. The solution of claim 21, wherein the non-ionic surfactant is glycerol-polyethylene glycol oxystearate.

35. The solution of claim 21, wherein the non-ionic surfactant is macrogolglycerol ricinoleate, macrogolglycerol hydroxystearate, polyethylene glycol 660 hydroxystearate, or a mixture thereof.

36. The solution of claim 21, wherein the non-ionic surfactant is polyethylene glycol 660 hydroxystearate.

37. The solution of claim 21, wherein the formulation is an oral formulation.

38. The solution of claim 37, wherein the oral formulation is a beverage.

39. The solution of claim 37, wherein the oral formulation is a spray or a tablet.

40. The solution of claim 37, wherein the oral formulation is present in a soft gel capsule, and the water content is less than about 10 wt %.

41. The solution of claim 21, wherein the formulation is a mucosal, parenteral, ocular, or topical formulation.

42. The solution of claim 21, wherein the dietary fatty acid is derived from a fish, algae, or vegetable source.

43. The solution of claim 21, further comprising a pharmaceutically acceptable excipient or stabilizer.

44. The solution of claim 21, consisting essentially of the dietary fatty acid, the non-ionic surfactant, and the water.

45. A method of delivering a dietary fatty acid to a subject, comprising administering the formulation of claim 21 to a subject such that the dietary fatty acid is more bioavailable than when the same amount of dietary fatty acid is delivered alone.

46. The method of claim 45, wherein the step of administering is by oral, mucosal, ocular, parenteral, or topical delivery.

47. The method of claim 45, wherein the administering is a result of the subject being treated for cancer, obesity, diabetes, cardiovascular disease, dyslipidaemia, age-related macular degeneration, high cholesterol, retinopathy, or a neurological disease.

48. A method of dissolving dietary fatty acids in water, comprising the steps of:
combining a dietary fatty acid with a warm, well mixed non-ionic surfactant to form a surfactant-dietary fatty acid mixture; and
continuously mixing the surfactant-dietary fatty acid mixture with water at least as slowly as necessary to solubilize the dietary fatty acid.

49. The method of claim 48, wherein said non-ionic surfactant is a glycerol-polyethylene glycol oxystearate, ethoxylated castor oil, polyethylene glycol 660 hydroxystarate, or a mixture thereof.

50. The method of claim 48, wherein the warm, well mixed non-ionic surfactant is prepared by the preliminary step of heating the surfactant to a temperature of about 90° F. to about 200° F. while mixing until clear.

51. The method of claim 48, wherein the combining step includes adding the dietary fatty acid to the non-ionic surfactant slowly and stirring until thoroughly mixed so as to constitute from 1 wt % to 75 wt % dietary fatty acid and from 25 wt % to 99 wt % surfactant, wherein the dietary fatty acid is sufficiently dispersed or dissolved in the surfactant so that the gel composition contains no visible micelles or particles of dietary fatty acid.

52. The method of claim 48, wherein the mixing step includes slowly adding the surfactant-dietary fatty acid mixture to warm water at a rate not to exceed 5 vol % of the water per second.

53. A method as in claim 48, wherein the step of heating the water-soluble non-ionic surfactant includes the step of stirring or mixing during the heating step.

54. A method of enhancing the bioavailability of a dietary fatty acid in a subject, said method comprising dissolving a surfactant-dietary fatty acid mixture in water as in claim 48.
